SEQUENCE LISTING (90 sequences)

SEQ ID NO: 1 - DNA, 679 bp, Zea mays
  source 1..679: mol_type = genomic DNA
tgctgttgaatgtgtctggtttgtatctctaatttctgatttcatgaattggtttacatgtcttacgaacctaacaccctttcactggtttgccattatgttnnnattgatatagcacatgtgtctttcatataatctttgctgaacattgctcttgaccaacatggttaacaggtctgacggggaaatggtattcgtggacgataagcagttgaaagctaaggagcccctggtggtatgacctcattgttttctcatttcacataatgaagtaaaaatggaatgacgccacaacaattattattctggtttgcagcttattgattactatgagcaacaccttcgctayaaccccacctcgtgataaggatgcattgacgctgctgggtttcagttgcatgtgctgcacctatgtttggaacaaatggaaatgtatttaaatgtagcattttttgtgctagatgcatttgtagtattatttgagtaagtagataattagtaagtagccaatgctgagtgcaagtgaaacatgatagcatggtttgtcaaactaactgctcgatattnnnactnnnttcaaattnnnttattaatcgtgataatcatgnnnnnnnnnnnnngtcgatttggaccaatagtgnnnnnncgnnnangtnannnnnnnaannnnnnnnnntna

SEQ ID NO: 2 - DNA, 522 bp, Zea mays
  source 1..522: mol_type = genomic DNA
tgtcttcctcgacggactttcattggacggcacccttattgtcaatgctgtggatggagcagaggtattatgacattaagcactgtcaaacccagagtcctggcataacacagatgcatgcctcggttcggttactaattaacttaaccatggaatatttttctcatgctacaaactgttcatgacctgctgcttgcaggttaaagttactggtcatgtagaaaataagggctggactatccggcatgtggactacaaggacacctcggagaaggaagaggtcgggatccgtgggttcaagttcracaagtctgagcagctagaggtgaactacaccgggccaggaaagcatagcttgagtgcgtcaaaagaaaacactcttgacttgcatgcttgacctgactagcttcgtacaaaaatagaaatagctcacatctgggtcgcctcccagattctgggagggaggccattgccagcgtagctgaaaccgttcttctccttcccccacgtacaacaaggagc

SEQ ID NO: 3 - DNA, 759 bp, Zea mays
  source 1..759: mol_type = genomic DNA
ctgctgttgccggcttcnncatgcagaaggtaataaaatgtcacccatgctcatgatcttttaagtgtatttaggtactgttaagtaggatggggtaaatattttntacctttcaaaagagaccaataaatctttttaacatttcagttttttcataaatnaaaaaaggaaagntttgacaaatgactttattatctcaggaacttgactaccttgttggagctgttgccaacccaaagaagccatttgccgcyattgttggtggatccaaggtctcaantaagattggtgtcattgagtcnttgctggcgaaggtcgatatcctcatccttggtggtggtatgatctacacattttacaaggcacanggatattctgttggaaaatctctcgtggaagaggataaactngagctcgcaacttctcttattgagaaggcgaaggcaaagggggtttctcttttgcttcccactgatattgtagtagcggacaagtttgcagcngatgctgagagcaaggtttgtttatttacacataaacacctcagatggnacttcagaattntttctttctttccctttaatgaatcgtgaannnnnnnnnnnagnnnnnctnnnnnnnnnnattcactagtaattctgccgcataagtcgtgcagcagantagaattaggcatgaatagttcagtgttaatagtgataagacatgttattttgtatgataggttttccacagtgcagnaccggaaacaaatnngnc

SEQ ID NO: 4 - DNA, 201 bp, Zea mays
  source 1..201: mol_type = genomic DNA
actaggatgcttccgatctgtttggatttgctaactttctagaaancatttaccacgagcgtaggtttcaacgttgaccacacaagaaagctggctgagcwtgagatcagccaccccatgtggctcattaaccagttagcatcttcgtatatgacttaaaggccntatgtggggtactccataactatttcccagctccca

SEQ ID NO: 5 - DNA, 201 bp, Zea mays
  source 1..201: mol_type = genomic DNA
gcatatcacttttagcaccttatagcactcgggtttggatctctatatgctaaaatctagctaaaagtgtagtgctaaacattagcaccatagattcaaasaagcccttaagcttaggactggttacttgaggtgtactttgagatcacaagggatcctagggtttgcacttggcaatcttgagtcctagcggttttagag

SEQ ID NO: 6 - DNA, 201 bp, Zea mays
  source 1..201: mol_type = genomic DNA
gaggtgtactttgagatcacaagggatcctagggtttgcacttggcaatcttgagtcctagcggttttagagttgtattgtcaaccctatcaaagcccttstgaagcaatatatgaggactaagtngggttgtccgcctnagggcctatttggatccaaggagctaaaacaaaactgactaaagtttantcactttagggc

SEQ ID NO: 7 - DNA, 121 bp, Zea mays
  source 1..121: mol_type = genomic DNA
ggaggtggcatcgattgnttgctcgnngatcacaagtcagatgcttcctgttgggagcctrgcttactgaagatcctcaaaatgcctttaccttgctatatgcaaatgtgtttcgggtatg

SEQ ID NO: 8 - DNA, 201 bp, Zea mays
  source 1..201: mol_type = genomic DNA
aagaaggcaactcancattcacatcgtctcgtgactccaataatnagggttagttggtcgtgaacatctgaaggtcaaatcaactagtccattaagttagsctggaagaatcaaagttgaatcaaagaggctatgaagaaaaaaaacccgatnaaccanccttgtttttcttgttagcttctttttgaatntcgagggcga

SEQ ID NO: 9 - DNA, 201 bp, Zea mays
  source 1..201: mol_type = genomic DNA
ccggaacatttgcaaaaatgagaggagtcacgcttcncctaaacctagagaaatgtgtctttggagttcaggcaaaaaattagggtactttgtgtcactcrgagggattaaagccaacccanacaagatacaagcaataataaacatgtaaccccnaagtcancaaaagncntctagaggttaataggctggctcacagnc

SEQ ID NO: 10 - DNA, 201 bp, Zea mays
  source 1..201: mol_type = genomic DNA
ctcaactttgtcatcaccctctagggcaattggtaagggtggaggcatctgctgagccactccaccccatcgtgtcgatcccagctccccggcatacaagmagcggaagttcaacatcgaagaagcctcttgataatgttcctatgggacatgacgaacattcttgagctcctcagcggggccctaaaataccactcttca

SEQ ID NO: 11 - DNA, 201 bp, Zea mays
  source 1..201: mol_type = genomic DNA
ttcttgttcgttcgatcatcctattatttagctactcatccatcatcatgttcagctcgtttaagacccgctagaccaatatctcagtcatataaactctrgtgactgatttttcttctagtaagaantttccagttggtcacccatccgagaattgctccaagttgtgcacgcttaactttgaggttctttngaacnagg

SEQ ID NO: 12 - DNA, 201 bp, Zea mays
  source 1..201: mol_type = genomic DNA
gtccgcagatgaatccatagncacaaacacccccaaccactccaatagatatatatggtgttaaccaaacacctaatatacgaggatagcaaaagactcastatggaaatgtgaaccacgtcatcgaagccacttaggatggcanaacaatattactaggtacactccaaatcaatgcacaccctnttgtcatactttttg

SEQ ID NO: 13 - DNA, 121 bp, Zea mays
  source 1..121: mol_type = genomic DNA
gataagaataaccgaacaagcccgaggtggcgcaagttgaggttggggacgaggtcgacgragggcagcntgagcatggtgancgggcacttgatgttgctggagtcnagcaagctggatg

SEQ ID NO: 14 - DNA, 201 bp, Zea mays
  source 1..201: mol_type = genomic DNA
gaaatcaattttaattttaatatgttttaattatctattgattatctataaacttgtacttaaataaaaattcacatatacatatgttaaatttagggtaygtcgtttgagttgaattgagtaaacatccgaacaagaattcgaattcaactatctattttgtattcatattctaatatattcgaatttgtatctgaattc

SEQ ID NO: 15 - DNA, 201 bp, Zea mays
  source 1..201: mol_type = genomic DNA
antcctcaaacctattcataacnncnctagtganggccctngcaaccctngtctcgaanatntataacatgtccgttgtttttggcccctacaagttactraaatcaacaagagatacaccaacaaatgtaggttattatacaaccaagtcacanaccttgctaattaacgtctctttcgattctttcatcttcctgctat

SEQ ID NO: 16 - DNA, 121 bp, Zea mays
  source 1..121: mol_type = genomic DNA
agtttggctagtgaagatcagaagaatggccctaggaacgttcttatgtgataacccttgytggatttcactgttgggatcctgatgtactttaaggcctgagacccccagaaatggatac

SEQ ID NO: 17 - DNA, 201 bp, Zea mays
  source 1..201: mol_type = genomic DNA
acctgcattcggacgtcgtnaagctcctgatgagnntgccttgtattgacctcaacatctgcgacaaggacggcatgactcctctggatctactcaggaarcagccacggacgacggcgtcagagntactgatcaaacaactgatcctggccggtgggttcacaagctcaagggaccacgagactaggtcggctatcgctt

SEQ ID NO: 18 - DNA, 698 bp, Zea mays
  source 1..698: mol_type = genomic DNA
taagatagaaagcaggagtcaattctgaataacactaggagctaagtctgacaatgtatatattttgaaggtaaaagatttattaaatatgagtagcttcgtagctgggagagtgatcattctcataatgaaacatggaactagtattgtatcatgataagcaataagattagaataattgaagaaagatcatacaaatgctccaggagcccattctcgatatgtgataccttccgcgctacattggaanataagatacaaagttatttgctagtatataggataaaagataaacacttcatgttaaagaatacctgcgattaaatccaaacttctcataactacgggagaaggcttccaagcctccttcatgttcatcaatgtctgaacggattcttctatagaggctgtacctgttcttttattcaaggaagttgaaagcacagtgagatggatagtggtgttcgcatgggttatatataggamtccttataagaagaatacagaagggatggaaatgaaggttgccaaagaattagaacccgctgttgcctctatattgtctgnctaacatttctgtaaatatcctctgaangagnacatatcttgtncgttcaanngtaatgcnnnntgaaaccannnnnnntacttnnnncatcaaagcagtatgnaagatttcnngtgaaaacgnnnctgnc

SEQ ID NO: 19 - DNA, 27 bp, synthetic construct
  source 1..27: mol_type = other DNA
tggtttgcagcttattgattactatga

SEQ ID NO: 20 - DNA, 21 bp, synthetic construct
  source 1..21: mol_type = other DNA
gacacctcggagaaggaagag

SEQ ID NO: 21 - DNA, 20 bp, synthetic construct
  source 1..20: mol_type = other DNA
gctgttgccaacccaaagaa

SEQ ID NO: 22 - DNA, 24 bp, synthetic construct
  source 1..24: mol_type = other DNA
gtttcaacgttgaccacacaagaa

SEQ ID NO: 23 - DNA, 32 bp, synthetic construct
  source 1..32: mol_type = other DNA
gctaaaatctagctaaaagtgtagtgctaaac

SEQ ID NO: 24 - DNA, 24 bp, synthetic construct
  source 1..24: mol_type = other DNA
gagtcctagcggttttagagttgt

SEQ ID NO: 25 - DNA, 24 bp, synthetic construct
  source 1..24: mol_type = other DNA
atcacaagtcagatgcttcctgtt

SEQ ID NO: 26 - DNA, 25 bp, synthetic construct
  source 1..25: mol_type = other DNA
tcgtgaacatctgaaggtcaaatca

SEQ ID NO: 27 - DNA, 28 bp, synthetic construct
  source 1..28: mol_type = other DNA
cctaaacctagagaaatgtgtctttgga

SEQ ID NO: 28 - DNA, 18 bp, synthetic construct
  source 1..18: mol_type = other DNA
ccaccccatcgtgtcgat

SEQ ID NO: 29 - DNA, 25 bp, synthetic construct
  source 1..25: mol_type = other DNA
cccgctagaccaatatctcagtcat

SEQ ID NO: 30 - DNA, 30 bp, synthetic construct
  source 1..30: mol_type = other DNA
gtgttaaccaaacacctaatatacgaggat

SEQ ID NO: 31 - DNA, 18 bp, synthetic construct
  source 1..18: mol_type = other DNA
gaggtggcgcaagttgag

SEQ ID NO: 32 - DNA, 35 bp, synthetic construct
  source 1..35: mol_type = other DNA
acttgtacttaaataaaaattcacatatacatatg

SEQ ID NO: 33 - DNA, 28 bp, synthetic construct
  source 1..28: mol_type = other DNA
gcaaccctagtctcgaaaatgtataaca

SEQ ID NO: 34 - DNA, 24 bp, synthetic construct
  source 1..24: mol_type = other DNA
gccctaggaacgttcttatgtgat

SEQ ID NO: 35 - DNA, 20 bp, synthetic construct
  source 1..20: mol_type = other DNA
acggcatgactcctctggat

SEQ ID NO: 36 - DNA, 25 bp, synthetic construct
  source 1..25: mol_type = other DNA
cacagtgagatggatagtggtgttc

SEQ ID NO: 37 - DNA, 21 bp, synthetic construct
  source 1..21: mol_type = other DNA
gcagcgtcaatgcatccttat

SEQ ID NO: 38 - DNA, 21 bp, synthetic construct
  source 1..21: mol_type = other DNA
cgcactcaagctatgctttcc

SEQ ID NO: 39 - DNA, 19 bp, synthetic construct
  source 1..19: mol_type = other DNA
tatcgaccttcgccagcaa

SEQ ID NO: 40 - DNA, 24 bp, synthetic construct
  source 1..24: mol_type = other DNA
gctaactggttaatgagccacatg

SEQ ID NO: 41 - DNA, 26 bp, synthetic construct
  source 1..26: mol_type = other DNA
gtacacctcaagtaaccagtcctaag

SEQ ID NO: 42 - DNA, 26 bp, synthetic construct
  source 1..26: mol_type = other DNA
agtcagttttgttttagctccttgga

SEQ ID NO: 43 - DNA, 25 bp, synthetic construct
  source 1..25: mol_type = other DNA
agcaaggtaaaggcattttgaggat

SEQ ID NO: 44 - DNA, 31 bp, synthetic construct
  source 1..31: mol_type = other DNA
attcaaaaagaagctaacaagaaaaacaagg

SEQ ID NO: 45 - DNA, 24 bp, synthetic construct
  source 1..24: mol_type = other DNA
tgagccagcctattaacctctaga

SEQ ID NO: 46 - DNA, 27 bp, synthetic construct
  source 1..27: mol_type = other DNA
ccataggaacattatcaagaggcttct

SEQ ID NO: 47 - DNA, 21 bp, synthetic construct
  source 1..21: mol_type = other DNA
ggatgggtgaccaactggaaa

SEQ ID NO: 48 - DNA, 21 bp, synthetic construct
  source 1..21: mol_type = other DNA
gcttcgatgacgtggttcaca

SEQ ID NO: 49 - DNA, 21 bp, synthetic construct
  source 1..21: mol_type = other DNA
gactccagcaacatcaagtgc

SEQ ID NO: 50 - DNA, 27 bp, synthetic construct
  source 1..27: mol_type = other DNA
tcgaattcttgttcggatgtttactca

SEQ ID NO: 51 - DNA, 31 bp, synthetic construct
  source 1..31: mol_type = other DNA
tgtgacttggttgtataataacctacatttg

SEQ ID NO: 52 - DNA, 25 bp, synthetic construct
  source 1..25: mol_type = other DNA
ggtctcaggccttaaagtacatcag

SEQ ID NO: 53 - DNA, 21 bp, synthetic construct
  source 1..21: mol_type = other DNA
cggccaggatcagttgtttga

SEQ ID NO: 54 - DNA, 25 bp, synthetic construct
  source 1..25: mol_type = other DNA
cttcatttccatcccttctgtattc

SEQ ID NO: 55 - DNA, 13 bp, synthetic construct
  source 1..13: mol_type = other DNA
ttcgctacaaccc

SEQ ID NO: 56 - DNA, 18 bp, synthetic construct
  source 1..18: mol_type = other DNA
ttcaagttcaacaagtct

SEQ ID NO: 57 - DNA, 15 bp, synthetic construct
  source 1..15: mol_type = other DNA
atttgccgccattgt

SEQ ID NO: 58 - DNA, 17 bp, synthetic construct
  source 1..17: mol_type = other DNA
ctggctgagcttgagat

SEQ ID NO: 59 - DNA, 19 bp, synthetic construct
  source 1..19: mol_type = other DNA
cttaagggcttgtttgaat

SEQ ID NO: 60 - DNA, 18 bp, synthetic construct
  source 1..18: mol_type = other DNA
caaagcccttctgaagca

SEQ ID NO: 61 - DNA, 18 bp, synthetic construct
  source 1..18: mol_type = other DNA
ttcagtaagctaggctcc

SEQ ID NO: 62 - DNA, 20 bp, synthetic construct
  source 1..20: mol_type = other DNA
ttgattcttccaggctaact

SEQ ID NO: 63 - DNA, 16 bp, synthetic construct
  source 1..16: mol_type = other DNA
tgtcactcggagggat

SEQ ID NO: 64 - DNA, 20 bp, synthetic construct
  source 1..20: mol_type = other DNA
ttgaacttccgcttcttgta

SEQ ID NO: 65 - DNA, 20 bp, synthetic construct
  source 1..20: mol_type = other DNA
aaaatcagtcactagagttt

SEQ ID NO: 66 - DNA, 20 bp, synthetic construct
  source 1..20: mol_type = other DNA
agcaaaagactcagtatgga

SEQ ID NO: 67 - DNA, 15 bp, synthetic construct
  source 1..15: mol_type = other DNA
ctgccctccgtcgac

SEQ ID NO: 68 - DNA, 19 bp, synthetic construct
  source 1..19: mol_type = other DNA
aactcaaacgacgtaccct

SEQ ID NO: 69 - DNA, 19 bp, synthetic construct
  source 1..19: mol_type = other DNA
ctacaagttactaaaatca

SEQ ID NO: 70 - DNA, 16 bp, synthetic construct
  source 1..16: mol_type = other DNA
aacccttgctggattt

SEQ ID NO: 71 - DNA, 16 bp, synthetic construct
  source 1..16: mol_type = other DNA
ctcaggaagcagccac

SEQ ID NO: 72 - DNA, 21 bp, synthetic construct
  source 1..21: mol_type = other DNA
ataaggattcctatatataac

SEQ ID NO: 73 - DNA, 14 bp, synthetic construct
  source 1..14: mol_type = other DNA
cttcgctataaccc

SEQ ID NO: 74 - DNA, 16 bp, synthetic construct
  source 1..16: mol_type = other DNA
caagttcgacaagtct

SEQ ID NO: 75 - DNA, 14 bp, synthetic construct
  source 1..14: mol_type = other DNA
ttgccgctattgtt

SEQ ID NO: 76 - DNA, 16 bp, synthetic construct
  source 1..16: mol_type = other DNA
tggctgagcatgagat

SEQ ID NO: 77 - DNA, 19 bp, synthetic construct
  source 1..19: mol_type = other DNA
cttaagggcttctttgaat

SEQ ID NO: 78 - DNA, 16 bp, synthetic construct
  source 1..16: mol_type = other DNA
aagcccttgtgaagca

SEQ ID NO: 79 - DNA, 15 bp, synthetic construct
  source 1..15: mol_type = other DNA
agtaagccaggctcc

SEQ ID NO: 80 - DNA, 20 bp, synthetic construct
  source 1..20: mol_type = other DNA
ttgattcttccagcctaact

SEQ ID NO: 81 - DNA, 16 bp, synthetic construct
  source 1..16: mol_type = other DNA
tgtcactcagagggat

SEQ ID NO: 82 - DNA, 16 bp, synthetic construct
  source 1..16: mol_type = other DNA
acttccgctgcttgta

SEQ ID NO: 83 - DNA, 15 bp, synthetic construct
  source 1..15: mol_type = other DNA
cagtcaccagagttt

SEQ ID NO: 84 - DNA, 20 bp, synthetic construct
  source 1..20: mol_type = other DNA
agcaaaagactcactatgga

SEQ ID NO: 85 - DNA, 15 bp, synthetic construct
  source 1..15: mol_type = other DNA
ctgcccttcgtcgac

SEQ ID NO: 86 - DNA, 17 bp, synthetic construct
  source 1..17: mol_type = other DNA
ctcaaacgacataccct

SEQ ID NO: 87 - DNA, 17 bp, synthetic construct
  source 1..17: mol_type = other DNA
acaagttactgaaatca

SEQ ID NO: 88 - DNA, 16 bp, synthetic construct
  source 1..16: mol_type = other DNA
aacccttgttggattt

SEQ ID NO: 89 - DNA, 17 bp, synthetic construct
  source 1..17: mol_type = other DNA
actcaggaaacagccac

SEQ ID NO: 90 - DNA, 21 bp, synthetic construct
  source 1..21: mol_type = other DNA
ataaggagtcctatatataac

(19) United States
(12) Patent Application Publication
(10) Pub. No.: US 2024/0237601 A1
(43) Pub. Date: Jul. 18, 2024

(54) HEAD SMUT RESISTANCE QTL

(71) Applicant: Monsanto Technology LLC, St. Louis, MO (US)

(72) Inventors: Juan Manuel Cruz Monasterio, San Pedro Tlaquepaque (MX); Jose Jaime Elizarraras, San Pedro Tlaquepaque (MX); Miguel Angel Gutierrez Gaitan, Edinburg, TX (US); Yule Pan, Chesterfield, MO (US); Jeanette Marie Peevers, Chesterfield, MO (US); Jose Hernan Santa Cruz Hidalgo, St. Louis, MO (US)

(21) Appl. No.: 18/533,009

(22) Filed: Dec. 7, 2023

Related U.S. Application Data
(60) Provisional application No. 63/386,802, filed on Dec. 9, 2022.

(51) Int. Cl. A01H 1/00; C12Q 1/6895

(52) U.S. Cl. CPC A01H 1/1255; C12Q 1/6895; C12Q 2600/13

(57) ABSTRACT

Corn plants exhibiting resistance to Head Smut disease are provided, together with methods of producing, identifying, or selecting plants or germplasm with a Head Smut resistance phenotype. Such methods include producing a corn plant exhibiting resistance to HS, comprising introgressing genomic regions conferring disease resistance; or selecting a corn plant exhibiting resistance to HS. Compositions, including polymorphic markers for detecting plants comprising introgressed disease resistance alleles, are further provided.

### CROSS REFERENCE TO RELATED APPLICATIONS

This application claims the benefit of U.S. Provisional Patent Appl. Ser. No. 63/386,802, filed Dec. 9, 2022, the entire disclosure of which is incorporated herein by reference.

### INCORPORATION OF SEQUENCE LISTING

A sequence listing containing the file named “MONS560US_ST26.xml” which is 83 kilobytes (measured in MS-Windows®) and created on Dec. 7, 2023, and comprises 90 sequences, is incorporated herein by reference in its entirety.

### FIELD OF THE INVENTION

The present invention relates to the field of plant breeding and more specifically to methods and compositions for producing corn plants exhibiting improved resistance to Head Smut.

### BACKGROUND

Head Smut (HS) is caused by the fungus Sphacelotheca reiliana. The fungus infects corn plants during the early vegetative stages and grows systemically in the plant. Symptoms are not evident until plants reach reproductive stages. Infected cars and tassels are replaced by smut sori (spore masses) filled with teliospores. Large amounts of teliospores are produced on the car and sometimes the tassel of infected plants. Yield loss due to HS disease is directly dependent upon the incidence of disease. The disease can also impact marketability as corn exhibiting HS disease is subject to export restrictions in some countries. Corn is the second most plentiful cereal grown for human consumption and the primary feed grain in the United States. Thus, resistance to HS is a particularly important trait for the production of corn. Although some HS resistance alleles have been identified, the mapping and introduction of sustainable resistance to pathogenic fungi remains one of the main challenges of modern plant breeding, especially in corn. Therefore, a continuing need exists in the art to identify new resistance alleles conferring increased resistance to HS as well as more effective methods of introgressing those resistance alleles into commercial lines to provide new varieties with improved resistance to HS infection.

### SUMMARY

In one aspect, provided herein is a method of producing a corn plant exhibiting resistance to Head Smut disease, comprising introgressing into a plant a Head Smut resistance allele within a chromosomal segment flanked in the genome of said plant by marker locus M2 (SEQ ID NO:2) and marker locus M13 (SEQ ID NO:13) on chromosome 5; wherein said introgressed Head Smut resistance allele confers to said plant increased resistance to Head Smut compared to a plant not comprising said allele. In some embodiments, the introgressing comprises: a) crossing a corn plant comprising said chromosomal segment with itself or with a second corn plant to produce one or more progeny plants; and b) selecting a progeny plant comprising said chromosomal segment. In further embodiments, the corn plant is homozygous for said Head Smut resistance allele; or the corn plant is heterozygous for said Head Smut resistance allele. In some embodiments, a representative the progeny plant is an F₁ hybrid plant. In other embodiments, a representative sample of seed comprising said chromosomal segment has been deposited under NCMA Accession No. 202209001. In certain embodiments, the Head Smut resistance allele is further defined as located within a chromosomal segment flanked in the genome of said plant by marker locus M3 (SEQ ID NO:3) and marker locus M10 (SEQ ID NO:10) on chromosome 5. In still further embodiments, the chromosomal segment comprises a marker locus on chromosome 5 selected from the group consisting of marker locus M2 (SEQ ID NO:2), M3 (SEQ ID NO:3), M4 (SEQ ID NO:4), M5 (SEQ ID NO:5), M6 (SEQ ID NO:6), M7 (SEQ ID NO:7), M8 (SEQ ID NO:8), M9 (SEQ ID NO:9), M10 (SEQ ID NO:10), M11 (SEQ ID NO:11), marker locus M12 (SEQ ID NO:12), and M13 (SEQ ID NO:13). In other embodiments, the introgressing comprises backcrossing, marker-assisted selection, or assaying for said Head Smut resistance.

In another aspect, methods are provided for selecting a corn plant with increased resistance to Head Smut disease comprising: (a) crossing a corn plant comprising a Head Smut resistance allele with a second corn plant to produce a population of progeny plants; and (b) selecting a progeny plant comprising said Head Smut resistance allele; wherein selecting said progeny plant comprises detecting a marker locus within or genetically linked to a chromosomal segment flanked in the genome of said plant by marker locus M2 (SEQ ID NO:2) and marker locus M13 (SEQ ID NO:13) on chromosome 5. In some embodiments, selecting said progeny plant is further defined as detecting a marker locus within or genetically linked to a chromosomal segment flanked in the genome of said plant by marker locus M3 (SEQ ID NO:3) and marker locus M10 (SEQ ID NO:10) on chromosome 5. In some embodiments, selecting a progeny plant comprises detecting nucleic acids comprising marker locus M1 (SEQ ID NO:1), M2 (SEQ ID NO:2), M3 (SEQ ID NO:3), M4 (SEQ ID NO:4), M5 (SEQ ID NO:5), M6 (SEQ ID NO:6), M7 (SEQ ID NO:7), M8 (SEQ ID NO:8), M9 (SEQ ID NO:9), M10 (SEQ ID NO: 10), M11 (SEQ ID NO:11), M12 (SEQ ID NO:12), M13 (SEQ ID NO:13), M14 (SEQ ID NO:14), M15 (SEQ ID NO:15), M16 (SEQ ID NO:16), M17 (SEQ ID NO:17), or marker locus M18 (SEQ ID NO:18). In still other embodiments, the progeny plant is an F₂-F₆ progeny plant or producing the progeny plant comprises backcrossing.

In a further aspect, methods are provided for selecting a corn plant exhibiting resistance to Head Smut, comprising: a) screening one or more plants with at least one nucleic acid marker to detect a polymorphism genetically linked to Head Smut resistance; and b) selecting one or more plants comprising said polymorphism genetically linked to Head Smut resistance, wherein said polymorphism is within or genetically linked to a chromosomal segment flanked in the genome of said plant by marker locus M2 (SEQ ID NO:2) and marker locus M13 (SEQ ID NO:13) on chromosome 5. In some embodiments, selecting one or more plants comprises: (a) detecting a marker locus within or genetically linked to a chromosomal segment flanked in the genome of said plant by marker locus M2 (SEQ ID NO:2) and marker locus M13 (SEQ ID NO:13) on chromosome 5; (b) detecting a marker locus within or genetically linked to a chromosomal segment flanked in the genome of said plant by marker locus M3 (SEQ ID NO:3) and marker locus M10 (SEQ ID NO:10) on chromosome 5; or (c) detecting at least one polymorphism at a locus selected from the group consisting of marker locus M1 (SEQ ID NO:1), M2 (SEQ ID NO:2), M3 (SEQ ID NO:3), M4 (SEQ ID NO:4), M5 (SEQ ID NO:5), M6 (SEQ ID NO:6), M7 (SEQ ID NO:7), M8 (SEQ ID NO:8), M9 (SEQ ID NO:9), M10 (SEQ ID NO: 10), M11 (SEQ ID NO: 11), M12 (SEQ ID NO: 12), M13 (SEQ ID NO:13), M14 (SEQ ID NO:14), M15 (SEQ ID NO:15), M16 (SEQ ID NO:16), M17 (SEQ ID NO:17), and marker locus M18 (SEQ ID NO:18). In further embodiments, the screening one or more plants comprises PCR, single strand conformational polymorphism analysis, denaturing gradient gel electrophoresis, cleavage fragment length polymorphism analysis, TAQMAN assay, and/or DNA sequencing. In certain embodiments, the corn plant comprises said chromosomal segment on chromosome 5, a representative sample of seed comprising said chromosomal segment has been deposited under NCMA Accession No. 202209001.

In yet another aspect, methods are provided for identifying a corn plant comprising a Head Smut resistance allele: (a) obtaining nucleic acids from at least a first corn plant; and (b) identifying in said nucleic acids the presence of at least a first genetic marker indicative of the presence of a chromosomal segment flanked in the genome of said plant by marker locus M2 (SEQ ID NO:2) and marker locus M13 (SEQ ID NO: 13) on chromosome 5; and wherein said Head Smut resistance allele confers to said plant increased resistance to Head Smut compared to a plant not comprising said allele. In some embodiments, the identifying comprises detecting a marker genetically linked to marker locus M1 (SEQ ID NO:1), M2 (SEQ ID NO:2), M3 (SEQ ID NO:3), M4 (SEQ ID NO:4), M5 (SEQ ID NO:5), M6 (SEQ ID NO:6), M7 (SEQ ID NO:7), M8 (SEQ ID NO:8), M9 (SEQ ID NO:9), M10 (SEQ ID NO: 10), M11 (SEQ ID NO:11), M12 (SEQ ID NO:12), M13 (SEQ ID NO:13), M14 (SEQ ID NO:14), M15 (SEQ ID NO:15), M16 (SEQ ID NO:16), M17 (SEQ ID NO:17), or marker locus M18 (SEQ ID NO:18).

In yet a further aspect, methods are provided for selecting a corn plant exhibiting resistance to Head Smut disease, comprising: a) obtaining a population of progeny plants having a parent comprising resistance to Head Smut; b) screening said population with at least one nucleic acid marker to detect a polymorphism genetically linked to Head Smut resistance; and c) selecting from said population one or more progeny plants based on the presence of said polymorphism, wherein said polymorphism is within or genetically linked to a chromosomal segment flanked in the genome of said plant by marker locus M2 (SEQ ID NO:2) and marker locus M13 (SEQ ID NO:13) on chromosome 5. In some embodiments, the first polymorphism is further defined as located within a chromosomal segment flanked in the genome by marker locus M2 (SEQ ID NO:2) and marker locus M13 (SEQ ID NO:13) on chromosome 5. In further embodiments, the first polymorphism is selected from the group consisting of: marker locus M2 (SEQ ID NO:2), M3 (SEQ ID NO:3), M4 (SEQ ID NO:4), M5 (SEQ ID NO:5), M6 (SEQ ID NO:6), M7 (SEQ ID NO:7), M8 (SEQ ID NO:8), M9 (SEQ ID NO:9), M10 (SEQ ID NO:10), M11 (SEQ ID NO:11), marker locus M12 (SEQ ID NO:12), and M13 (SEQ ID NO:13).

### BRIEF DESCRIPTION OF THE DRAWINGS

FIG. 1: Shows a schematic overview of the Head Smut resistance QTL on the physical map of chromosomes 5.

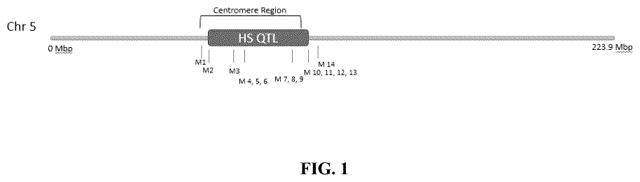

### DETAILED DESCRIPTION

Head smut is caused by the pathogenic fungus Sphacelotheca reiliana (also known as Sorosporium reilianum, Ustilago reiliana, Sporisorium holci-sorghi). Teliospores of S. reiliana are circular, spiny, and dark brown to black in color. Several races of S. reiliana have been recognized that affect hosts including corn (i.e. maize), sorghum, Sudan grass, Johnson grass, and teosinte. Head smut is a systemic disease in corn. The fungus infects corn plants during the early vegetative stages. However, plants infected with S. reiliana remain asymptomatic until the plant reaches its reproductive stage. Symptoms consist of conspicuous galls that replace cars or tassels. These galls are covered by a very fragile membrane that breaks early and easily, exposing masses of dark spores, teliospores, and vascular bundles. The consistency of the membrane and the presence of vascular bundles are what differentiates Head Smut from common smut. Once the membrane is broken, wind and rain spread the teliospores into neighboring soil where they can stay viable for several years. As such, soil-borne teliospores are the source of the mycelium that penetrates through the seedling root, invades the meristematic tissue, and develops systemically.

Common management practices to reduce the incidence and spread of Head Smut in corn include early planting when temperatures are unfavorable for spore germination, treating seeds with protective fungicides, maintaining balanced soil fertility, and removing and burning smutted tassels and cars as they emerge. Although some HS resistance alleles have been identified, the mapping and introduction of novel genetic loci associated with resistance to Head Smut disease would represent a significant benefit to both corn plant breeders as well as farmers.

The present inventors have made significant advancements in obtaining Head Smut resistance in corn by identifying a novel Head Smut resistance QTL on chromosome 5. The QTL disclosed herein is distinct from those known in the art. In addition, novel markers for the introgressed alleles are provided, allowing the alleles to be accurately introgressed and tracked during plant breeding. As such, the invention permits introgression of the disease resistance alleles into any desired corn genotype and provide valuable new tools for engineering HS resistance in corn.

As disclosed herein, M2, a SNP marker with a [A/G] change at 58,597,975 bp on chromosome 5 of the public maize genomic map IBM2 2008 Neighbors (available at https://www.maizegdb.org/), and M13, a SNP marker with a [G/A] change at 155,327,347 bp on chromosome 5 of the public maize genome map IBM2 2008 Neighbors, can be used to identify this region, wherein M2 and M13 are flanking markers for the chromosomal segment on chromosome 5. The public genome of maize is available at, e.g. Maize GDB (https://www.maizegdb.org/), or more specifically at https://www.maizegdb.org/gbrowse/maize_v4 (B73_REFGEN_V4), and one skilled in the art would understand that the marker sequences provided for the first time in the instant application could be located on any version (or later version) of the public genome. One aspect of the present disclosure therefore provides methods of producing a corn plant exhibiting resistance to Head Smut disease, comprising introgressing into a plant a Head Smut resistance allele within a chromosomal segment flanked in the genome of said plant by marker locus M2 (SEQ ID NO:2) and marker locus M13 (SEQ ID NO:13) on chromosome 5; wherein said introgressed Head Smut resistance allele confers to said plant increased resistance to Head Smut compared to a plant not comprising said allele. In other aspects, the Head Smut resistance allele is further defined as located within a chromosomal segment flanked in the genome of said plant by marker locus M3 (SEQ ID NO:3) and marker locus M10 (SEQ ID NO:10) on chromosome 5.

In another aspect, corn plants are provided that are obtainable by a method disclosed herein, wherein the plants comprise the HS resistance allele within a chromosomal segment flanked in the genome of the plant by marker locus M2 (SEQ ID NO:2) and marker locus M13 (SEQ ID NO:13) on chromosome 5. In particular embodiments, said plants are not exclusively obtained by means of an essentially biological process.

In some embodiments, provided herein are methods of selecting a corn plant exhibiting resistance to Head Smut disease. In certain embodiments, said methods comprise screening one or more plants with at least one nucleic acid marker to detect a polymorphism genetically linked to Head Smut resistance, and selecting one or more plants comprising said polymorphism genetically linked to Head Smut resistance. In further embodiments, said selecting comprising detecting a marker locus within or genetically linked to a chromosomal segment flanked in the genome of said plant by marker locus M2 (SEQ ID NO:2) and marker locus M13 (SEQ ID NO:13) on chromosome 5. In particular embodiments, selecting a corn plant exhibiting resistance to Head Smut disease comprises molecular genetic techniques. For example, those of ordinary skill in the art viewing the present disclosure may use technical methods to select a corn plant exhibiting resistance to Head Smut disease by screening one or more plants with at least one nucleic acid marker to detect a polymorphism genetically linked to HS resistance.

In some embodiments, the present disclosure provides the markers shown in Table 5, which have been shown to be genetically linked to Head Smut resistance in corn plants. In particular embodiments, plants comprising the Head Smut resistance alleles are provided. The HS resistance alleles described herein provide robust resistance to Head Smut disease. Methods of producing the plants described herein are further provided. The disclosure further provides trait-linked markers which can be used to produce and/or select plants comprising the HS resistance alleles on chromosome 5 conferring Head Smut resistance as described herein.

### I. Genomic Regions, Alleles, and Polymorphisms Associated with Head Smut Resistance in Corn Plants

Provided herein are introgressions of one or more alleles associated with Head Smut resistance, together with polymorphic nucleic acids and linked markers for tracking the introgressions during plant breeding.

Corn lines exhibiting Head Smut resistance are known in the art and may be used together with the trait-linked markers provided herein in accordance with the present disclosure. For example, corn line AM7739/CV890999 derived F1, comprising the chromosomal segment on chromosomes 5, wherein a representative sample of seed comprising the chromosomal segment has been deposited under NCMA Accession No. 202209001, can be used as a source for HS resistance according to the disclosure.

Using the improved genetic markers and the assays described herein, those skilled in the art are able to successfully produce and/or select plants comprising the Head Smut resistance alleles described herein, which confer increased resistance to HS as compared to a plant not comprising the allele(s). In certain embodiments, provided herein are methods of introgressing into a plant a Head Smut resistance allele within a chromosomal segment flanked in the genome of the plant by marker locus M2 (SEQ ID NO:2) and marker locus M13 (SEQ ID NO:13) on chromosome 5. The present disclosure therefore represents a significant advance in the art.

### II. Introgression of Genomic Regions Associated with Head Smut Resistance

Marker-assisted introgression involves the transfer of a chromosomal region defined by one or more markers from a first genetic background to a second. Offspring of a cross that contain the introgressed genomic region can be identified by the combination of markers characteristic of the desired introgressed genomic region from a first genetic background and both linked and unlinked markers characteristic of the second genetic background.

Provided herein are accurate markers for identifying and tracking introgression of one or more of the genomic regions disclosed herein from a Head Smut resistant plant into a cultivated line. Further provided are markers for identifying and tracking the introgressions disclosed herein during plant breeding, including the markers set forth in Table 5.

Markers within or linked to any of the genomic intervals described herein may be useful in a variety of breeding efforts that include introgression of genomic regions associated with disease resistance into a desired genetic background. For example, a marker within 40 cM, 20 cM, 15 cM, 10 cM, 5 cm, 2 cM, or 1 cM of a marker associated with disease resistance described herein can be used for marker-assisted introgression of genomic regions associated with a disease resistant phenotype.

Methods of producing, selecting, and identifying corn plants comprising one or more introgressed regions associated with a desired phenotype wherein at least 10%, 25%, 50%, 75%, 90%, or 99% of the remaining genomic sequences carry markers characteristic of the recurrent parent germplasm are also provided. Methods of producing, selecting, and identifying corn plants comprising an introgressed region comprising regions closely linked to or adjacent to the genomic regions and markers provided herein and associated with a disease resistance phenotype are also provided.

### III. Development of Disease Resistant Corn Varieties

For most breeding objectives, commercial breeders work within germplasm that is “cultivated,” “cultivated type,” or “elite.” These cultivated lines may be used as recurrent parents or as a source of recurrent parent alleles during breeding. Cultivated or elite germplasm is easier to breed because it generally performs well when evaluated for horticultural performance. Many cultivated corn types have been developed and are known in the art as being agronomically elite and appropriate for commercial cultivation. However, the performance advantage a cultivated germplasm provides can be offset by a lack of allelic diversity. Breeders generally accept this tradeoff because progress is faster when working with cultivated material than when breeding with genetically diverse sources.

In contrast, when cultivated germplasm is crossed with non-cultivated germplasm, a breeder can gain access to novel alleles from the non-cultivated type. Non-cultivated germplasm may be used as a source of donor alleles during breeding. However, this approach generally presents significant difficulties due to fertility problems associated with crosses between diverse lines, and negative linkage drag from the non-cultivated parent. For example, non-cultivated corn types can provide alleles associated with disease resistance. However, these non-cultivated types may have poor horticultural qualities, agronomically unacceptable plant architecture, and/or necrosis.

The process of introgressing desirable resistance genes from non-cultivated lines into elite cultivated lines while avoiding problems with linkage drag or low heritability is a long and often arduous process. In deploying alleles derived from wild relatives it is often desirable to introduce a minimal or truncated introgression that provides the desired trait but lacks detrimental effects. To aid introgression reliable marker assays are preferable to phenotypic screens. Success is furthered by simplifying genetics for key attributes to allow focus on genetic gain for quantitative traits such as disease resistance. Moreover, the process of introgressing genomic regions from non-cultivated lines can be greatly facilitated by the availability of accurate markers for MAS.

One of skill in the art would therefore understand that the alleles, polymorphisms, and markers provided by the present disclosure allow the tracking and introduction of any of the genomic regions identified herein into any genetic background. In addition, the genomic regions associated with disease resistance disclosed herein can be introgressed from one genotype to another and tracked using MAS. Thus, the disclosure of accurate markers associated with disease resistance will facilitate the development of corn plants having beneficial phenotypes. For example, seed can be genotyped using the markers of the present disclosure to select for plants comprising desired genomic regions associated with disease resistance. Moreover, MAS allows identification of plants homozygous or heterozygous for a desired introgression.

Inter-species crosses can also result in suppressed recombination and plants with low fertility or fecundity. For example, suppressed recombination has been observed for the tomato nematode resistance gene Mi, the Mla and Mlg genes in barley, the Yr17 and Lr20 genes in wheat, the Runl gene in grapevine, and the Rma gene in peanut. Meiotic recombination is essential for classical breeding because it enables the transfer of favorable alleles across genetic backgrounds, the removal of deleterious genomic fragments, and pyramiding traits that are genetically tightly linked. Therefore suppressed recombination forces breeders to enlarge segregating populations for progeny screens in order to arrive at the desired genetic combination.

Phenotypic evaluation of large populations is time-consuming, resource-intensive and not reproducible in every environment. Marker-assisted selection offers a feasible alternative. Molecular assays designed to detect unique polymorphisms, such as SNPs, are versatile. However, they may fail to discriminate alleles within and among corn species in a single assay. Structural rearrangements of chromosomes such as deletions impair hybridization and extension of synthetically labeled oligonucleotides. In the case of duplication events, multiple copies are amplified in a single reaction without distinction. The development and validation of accurate and highly predictive markers are therefore essential for successful MAS breeding programs.

### IV. Marker Assisted Breeding and Genetic Engineering Techniques

Genetic markers that can be used in the practice of the present invention include, but are not limited to, restriction fragment length polymorphisms (RFLPs), amplified fragment length polymorphisms (AFLPs), simple sequence repeats (SSRs), simple sequence length polymorphisms (SSLPs), single nucleotide polymorphisms (SNPs), insertion/deletion polymorphisms (Indels), variable number tandem repeats (VNTRs), and random amplified polymorphic DNA (RAPD), isozymes, and other markers known to those skilled in the art. Marker discovery and development in crop plants provides the initial framework for applications to marker-assisted breeding activities (U.S. Patent Pub. Nos.: 2005/0204780, 2005/0216545, 2005/0218305, and 2006/00504538). The resulting “genetic map” is the representation of the relative position of characterized loci (polymorphic nucleic acid markers or any other locus for which alleles can be identified) to each other.

Polymorphisms comprising as little as a single nucleotide change can be assayed in a number of ways. For example, detection can be made by electrophoretic techniques including a single strand conformational polymorphism (Orita, et al. (1989) Genomics, 8(2), 271-278), denaturing gradient gel electrophoresis (Myers (1985) EPO 0273085), or cleavage fragment length polymorphisms (Life Technologies, Inc., Gaithersburg, MD), but the widespread availability of DNA sequencing often makes it easier to simply sequence amplified products directly. Once the polymorphic sequence difference is known, rapid assays can be designed for progeny testing, typically involving some version of PCR amplification of specific alleles (PASA; Sommer, et al. (1992) Biotechniques 12(1), 82-87), or PCR amplification of multiple specific alleles (PAMSA; Dutton and Sommer (1991) Biotechniques, 11(6), 700-7002).

Polymorphic markers serve as useful tools for assaying plants for determining the degree of identity of lines or varieties (U.S. Pat. No. 6,207,367). These markers form the basis for determining associations with phenotypes and can be used to drive genetic gain. In certain embodiments, polymorphic nucleic acids can be used to detect in a corn plant a genotype associated with disease resistance, identify a corn plant with a genotype associated with disease resistance, and to select a corn plant with a genotype associated with disease resistance. In certain embodiments of methods described, polymorphic nucleic acids can be used to produce a corn plant that comprises in its genome an introgressed locus associated with disease resistance. In certain embodiments, polymorphic nucleic acids can be used to breed progeny corn plants comprising a locus or loci associated with disease resistance.

Genetic markers may include “dominant” or “codominant” markers. “Codominant” markers reveal the presence of two or more alleles (two per diploid individual). “Dominant” markers reveal the presence of only a single allele. Markers are preferably inherited in codominant fashion so that the presence of both alleles at a diploid locus, or multiple alleles in triploid or tetraploid loci, are readily detectable, and they are free of environmental variation, i.e., their heritability is 1. A marker genotype typically comprises two marker alleles at each locus in a diploid organism. The marker allelic composition of each locus can be either homozygous or heterozygous. Homozygosity is a condition where both alleles at a locus are characterized by the same nucleotide sequence. Heterozygosity refers to a condition where the two alleles at a locus are different.

Nucleic acid-based analyses for determining the presence or absence of the genetic polymorphism (i.e. for genotyping) can be used in breeding programs for identification, selection, introgression, and the like. A wide variety of genetic markers for the analysis of genetic polymorphisms are available and known to those of skill in the art. The analysis may be used to select for genes, portions of genes, QTL, alleles, or genomic regions that comprise or are linked to a genetic marker that is linked to or associated with disease resistance in corn plants.

As used herein, nucleic acid analysis methods include, but are not limited to, PCR-based detection methods (for example, TaqMan assays), microarray methods, mass spectrometry-based methods and/or nucleic acid sequencing methods, including whole genome sequencing. In certain embodiments, the detection of polymorphic sites in a sample of DNA, RNA, or cDNA may be facilitated through the use of nucleic acid amplification methods. Such methods specifically increase the concentration of polynucleotides that span the polymorphic site, or include that site and sequences located either distal or proximal to it. Such amplified molecules can be readily detected by gel electrophoresis, fluorescence detection methods, or other means.

One method of achieving such amplification employs the polymerase chain reaction (PCR) (Mullis et al. (1986) Cold Spring Harbor Symp. Quant. Biol. 51:263-273; European Patent 50,424; European Patent 84,796; European Patent 258,017; European Patent 237,362; European Patent 201,184; U.S. Pat. Nos. 4,683,202; 4,582,788; and 4,683,194), using primer pairs that are capable of hybridizing to the proximal sequences that define a polymorphism in its double-stranded form. Methods for typing DNA based on mass spectrometry can also be used. Such methods are disclosed in U.S. Pat. Nos. 6,613,509 and 6,503,710, and references found therein.

Polymorphisms in DNA sequences can be detected or typed by a variety of effective methods well known in the art including, but not limited to, those disclosed in U.S. Pat. Nos. 5,468,613, 5,217,863; 5,210,015; 5,876,930; 6,030,787; 6,004,744; 6,013,431; 5,595,890; 5,762,876; 5,945,283; 5,468,613; 6,090,558; 5,800,944; 5,616,464; 7,312,039; 7,238,476; 7,297,485; 7,282,355; 7,270,981 and 7,250,252 all of which are incorporated herein by reference in their entirety. However, the compositions and methods of the present disclosure can be used in conjunction with any polymorphism typing method to detect polymorphisms in genomic DNA samples. These genomic DNA samples used include but are not limited to, genomic DNA isolated directly from a plant, cloned genomic DNA, or amplified genomic DNA.

For instance, polymorphisms in DNA sequences can be detected by hybridization to allele-specific oligonucleotide (ASO) probes as disclosed in U.S. Pat. Nos. 5,468,613 and 5,217,863. U.S. Pat. No. 5,468,613 discloses allele specific oligonucleotide hybridizations where single or multiple nucleotide variations in nucleic acid sequence can be detected in nucleic acids by a process in which the sequence containing the nucleotide variation is amplified, spotted on a membrane and treated with a labeled sequence-specific oligonucleotide probe.

Target nucleic acid sequence can also be detected by probe ligation methods, for example as disclosed in U.S. Pat. No. 5,800,944 where sequence of interest is amplified and hybridized to probes followed by ligation to detect a labeled part of the probe.

Microarrays can also be used for polymorphism detection, wherein oligonucleotide probe sets are assembled in an overlapping fashion to represent a single sequence such that a difference in the target sequence at one point would result in partial probe hybridization (Borevitz et al., Genome Res. 13:513-523 (2003); Cui et al., Bioinformatics 21:3852-3858 (2005). On any one microarray, it is expected there will be a plurality of target sequences, which may represent genes and/or noncoding regions wherein each target sequence is represented by a series of overlapping oligonucleotides, rather than by a single probe. This platform provides for high throughput screening of a plurality of polymorphisms. Typing of target sequences by microarray-based methods is described in U.S. Pat. Nos. 6,799,122; 6,913,879; and 6,996,476.

Other methods for detecting SNPs and Indels include single base extension (SBE) methods. Examples of SBE methods include, but are not limited, to those disclosed in U.S. Pat. Nos. 6,004,744; 6,013,431; 5,595,890; 5,762,876; and 5,945,283.

In another method for detecting polymorphisms, SNPs and Indels can be detected by methods disclosed in U.S. Pat. Nos. 5,210,015; 5,876,930; and 6,030,787 in which an oligonucleotide probe having a 5′ fluorescent reporter dye and a 3′ quencher dye covalently linked to the 5′ and 3′ ends of the probe. When the probe is intact, the proximity of the reporter dye to the quencher dye results in the suppression of the reporter dye fluorescence, e.g. by Forster-type energy transfer. During PCR, forward and reverse primers hybridize to a specific sequence of the target DNA flanking a polymorphism while the hybridization probe hybridizes to polymorphism-containing sequence within the amplified PCR product. In the subsequent PCR cycle DNA polymerase with 5′→3′ exonuclease activity cleaves the probe and separates the reporter dye from the quencher dye resulting in increased fluorescence of the reporter.

In another embodiment, a locus or loci of interest can be directly sequenced using nucleic acid sequencing technologies. Methods for nucleic acid sequencing are known in the art and include technologies provided by 454 Life Sciences (Branford, CT), Agencourt Bioscience (Beverly, MA), Applied Biosystems (Foster City, CA), LI-COR Biosciences (Lincoln, NE), NimbleGen Systems (Madison, WI), Illumina (San Diego, CA), and VisiGen Biotechnologies (Houston, TX). Such nucleic acid sequencing technologies comprise formats such as parallel bead arrays, sequencing by ligation, capillary electrophoresis, electronic microchips, “biochips,” microarrays, parallel microchips, and single-molecule arrays.

Various genetic engineering technologies have been developed and may be used by those of skill in the art to introduce traits in plants. In certain aspects, traits are introduced into corn plants via altering or introducing a single genetic locus or transgene into the genome of a variety or progenitor thereof. Methods of genetic engineering to modify, delete, or insert genes and polynucleotides into the genomic DNA of plants are well-known in the art.

In specific embodiments, improved corn lines can be created through the site-specific modification of a plant genome. Methods of genetic engineering include, for example, utilizing sequence-specific nucleases such as zinc-finger nucleases (see, for example, U.S. Pat. Appl. Pub. No. 2011-0203012); engineered or native meganucleases; TALE-endonucleases (see, for example, U.S. Pat. Nos. 8,586,363 and 9,181,535); and RNA-guided endonucleases, such as those of the CRISPR/Cas systems (see, for example, U.S. Pat. Nos. 8,697,359 and 8,771,945 and U.S. Pat. Appl. Pub. No. 2014-0068797). One embodiment of the disclosure thus relates to utilizing a nuclease or any associated protein to carry out genome modification. This nuclease could be provided heterologously within donor template DNA for templated-genomic editing or in a separate molecule or vector. A recombinant DNA construct may also comprise a sequence encoding one or more guide RNAs to direct the nuclease to the site within the plant genome to be modified. Further methods for altering or introducing a single genetic locus include, for example, utilizing single-stranded oligonucleotides to introduce base pair modifications in a corn plant genome (see, for example Sauer et al., Plant Physiol, 170(4): 1917-1928, 2016).

Methods for site-directed alteration or introduction of a single genetic locus are well-known in the art and include those that utilize sequence-specific nucleases, such as the aforementioned, or complexes of proteins and guide-RNA that cut genomic DNA to produce a double-strand break (DSB) or nick at a genetic locus. As is well-understood in the art, during the process of repairing the DSB or nick introduced by the nuclease enzyme, a donor template, transgene, or expression cassette polynucleotide may become integrated into the genome at the site of the DSB or nick. The presence of homology arms in the DNA to be integrated may promote the adoption and targeting of the insertion sequence into the plant genome during the repair process through homologous recombination or non-homologous end joining (NHEJ).

In another embodiment of the present disclosure, genetic transformation may be used to insert a selected transgene into a plant or may, alternatively, be used for the preparation of transgenes which can be introduced by backcrossing. Methods for the transformation of plants that are well-known to those of skill in the art and applicable to many crop species include, but are not limited to, electroporation, microprojectile bombardment, Agrobacterium-mediated transformation, and direct DNA uptake by protoplasts.

To effect transformation by electroporation, one may employ either friable tissues, such as a suspension culture of cells or embryogenic callus or alternatively one may transform immature embryos or other organized tissue directly. In this technique, one would partially degrade the cell walls of the chosen cells by exposing them to pectin-degrading enzymes (pectolyases) or mechanically wound tissues in a controlled manner.

An efficient method for delivering transforming DNA segments to plant cells is microprojectile bombardment. In this method, particles are coated with nucleic acids and delivered into cells by a propelling force. Exemplary particles include those comprised of tungsten, platinum, and preferably, gold. For the bombardment, cells in suspension are concentrated on filters or solid culture medium. Alternatively, immature embryos or other target cells may be arranged on solid culture medium. The cells to be bombarded are positioned at an appropriate distance below the macroprojectile stopping plate.

An illustrative embodiment of a method for delivering DNA into plant cells by acceleration is the Biolistics Particle Delivery System, which can be used to propel particles coated with DNA or cells through a screen, such as a stainless steel or Nytex screen, onto a surface covered with target cells. The screen disperses the particles so that they are not delivered to the recipient cells in large aggregates. Microprojectile bombardment techniques are widely applicable, and may be used to transform virtually any plant species.

Agrobacterium-mediated transfer is another widely applicable system for introducing gene loci into plant cells. An advantage of the technique is that DNA can be introduced into whole plant tissues, thereby bypassing the need for regeneration of an intact plant from a protoplast. Modern Agrobacterium transformation vectors are capable of replication in E. coli as well as Agrobacterium, allowing for convenient manipulations (Klee et al., Nat. Biotechnol., 3(7):637-642, 1985). Moreover, recent technological advances in vectors for Agrobacterium-mediated gene transfer have improved the arrangement of genes and restriction sites in the vectors to facilitate the construction of vectors capable of expressing various polypeptide coding genes. The vectors described have convenient multi-linker regions flanked by a promoter and a polyadenylation site for direct expression of inserted polypeptide coding genes. Additionally, Agrobacterium containing both armed and disarmed Ti genes can be used for transformation.

In those plant strains where Agrobacterium-mediated transformation is efficient, it is the method of choice because of the facile and defined nature of the gene locus transfer. The use of Agrobacterium-mediated plant integrating vectors to introduce DNA into plant cells is well known in the art (Fraley et al., Nat. Biotechnol., 3:629-635, 1985; U.S. Pat. No. 5,563,055).

Transformation of plant protoplasts also can be achieved using methods based on calcium phosphate precipitation, polyethylene glycol treatment, electroporation, and combinations of these treatments (see, for example, Potrykus et al., Mol. Gen. Genet., 199:183-188, 1985; Omirullch et al., Plant Mol. Biol., 21(3):415-428, 1993; Fromm et al., Nature, 312:791-793, 1986; Uchimiya et al., Mol. Gen. Genet., 204:204, 1986; Marcotte et al., Nature, 335:454, 1988). Transformation of plants and expression of foreign genetic elements is exemplified in Choi et al. (Plant Cell Rep., 13:344-348, 1994), and Ellul et al. (Theor. Appl. Genet., 107:462-469, 2003).

### V. Definitions

The following definitions are provided to better define the present invention and to guide those of ordinary skill in the art in the practice of the present invention. Unless otherwise noted, terms are to be understood according to conventional usage by those of ordinary skill in the relevant art.

As used herein, the term “plant” includes plant cells, plant protoplasts, plant cells of tissue culture from which corn plants can be regenerated, plant calli, plant clumps and plant cells that are intact in plants or parts of plants such as pollen, flowers, seeds, leaves, stems, and the like. In certain embodiments, the plant part can be a non-regenerable portion of a plant part. As used in this context, a “non-regenerable” portion of a plant part is a portion that cannot be induced to form a whole plant or that cannot be induced to form a whole plant that is capable of sexual and/or asexual reproduction. In certain embodiments, a non-regenerable portion of a plant part is a portion of a seed, leaf, flower, stem, or root. In particular embodiments, the plant or part thereof can be a corn plant, corn variety, or corn hybrid plant.

As used herein, a “control” plant, plant seed, embryo, plant part, plant cell, and/or plant genome may also be a plant, plant seed, plant part, embryo, plant cell, and/or plant genome having a similar (but not the same or identical) genetic background to a modified plant, plant seed, plant part, plant cell, and/or plant genome, if deemed sufficiently similar for comparison of the characteristics or traits to be analyzed.

As used herein, the term “population” means a genetically heterogeneous collection of plants that share a common parental derivation.

As used herein, the terms “variety” and “cultivar” mean a group of similar plants that by their genetic pedigrees and performance can be identified from other varieties within the same species.

As used herein, an “allele” refers to one of two or more alternative forms of a genomic sequence at a given locus on a chromosome.

A “quantitative trait locus” (QTL) is a chromosomal location that encodes for at least a first allele that affects the expressivity of a phenotype.

As used herein, a “marker” means a detectable characteristic that can be used to discriminate between organisms. Examples of such characteristics include, but are not limited to, genetic markers, biochemical markers, metabolites, morphological characteristics, and agronomic characteristics.

As used herein, the term “phenotype” means the detectable characteristics of a cell or organism that can be influenced by gene expression.

As used herein, the term “genotype” means the specific allelic makeup of a plant.

As used herein, “elite” or “cultivated” variety means any variety that has resulted from breeding and selection for superior agronomic performance. An “elite plant” refers to a plant belonging to an elite variety. Numerous elite varieties are available and known to those of skill in the art of corn breeding. An “elite population” is an assortment of elite individuals or varieties that can be used to represent the state of the art in terms of agronomically superior genotypes of a given crop species, such as corn. Similarly, an “elite germplasm” or elite strain of germplasm is an agronomically superior germplasm.

As used herein, the term “introgressed,” when used in reference to a genetic locus, refers to a genetic locus that has been introduced into a new genetic background, such as through backcrossing. Introgression of a genetic locus can be achieved through plant breeding methods and/or by molecular genetic methods. Such molecular genetic methods include, but are not limited to, various plant transformation techniques and/or methods that provide for homologous recombination, non-homologous recombination, site-specific recombination, and/or genomic modifications that provide for locus substitution or locus conversion.

As used herein, the terms “recombinant” or “recombined” in the context of a chromosomal segment refer to recombinant DNA sequences comprising one or more genetic loci in a configuration in which they are not found in nature, for example as a result of a recombination event between homologous chromosomes during meiosis.

As used herein, the term “linked,” when used in the context of nucleic acid markers and/or genomic regions, means that the markers and/or genomic regions are located on the same linkage group or chromosome such that they tend to segregate together at meiosis.

As used herein, “tolerance locus” means a locus associated with tolerance or resistance to disease. For instance, a tolerance locus according to the present disclosure may, in one embodiment, control tolerance or susceptibility to Head Smut Disease.

As used herein, “tolerance” or “improved tolerance” in a plant refers to the ability of the plant to perform well, for example by maintaining yield, under disease conditions. Tolerance may also refer to the ability of a plant to maintain a plant vigor phenotype under disease conditions. Tolerance is a relative term, indicating that a “tolerant” plant is more able to maintain performance compared to a different (less tolerant) plant (e.g. a different plant variety) grown in similar disease conditions. One of skill will appreciate that plant tolerance to disease conditions varies widely, and can represent a spectrum of more-tolerant or less-tolerant phenotypes. However, by simple observation, one of skill can generally determine the relative tolerance of different plants, plant varieties, or plant families under disease conditions, and furthermore, will also recognize the phenotypic gradations of “tolerance.”

As used herein “resistance” or “improved resistance” in a plant to disease conditions is an indication that the plant is more able to reduce disease burden than a non-resistant or less resistant plant. Resistance is a relative term, indicating that a “resistant” plant is more able to reduce disease burden compared to a different (less resistant) plant (e.g., a different plant variety) grown in similar disease conditions. One of skill will appreciate that plant resistance to disease conditions varies widely, and can represent a spectrum of more-resistant or less-resistant phenotypes. However, by simple observation, one of skill can generally determine the relative resistance of different plants, plant varieties, or plant families under disease conditions, and furthermore, will also recognize the phenotypic gradations of “resistant.”

As used herein “reduced” or “reduction” in the context of Head Smut disease is an indication that the plant exhibits a decrease in one or more specific symptoms of Head Smut (e.g. rounded cars that do not produce silks, tassels completely or partially covered by sori, absence of pollen production, presence of head smut galls) or any combination thereof. Reduction is a relative term, indicating that a plant comprising “reduced Head Smut disease” exhibits less symptoms of Head Smut compared to a different plant (e.g., a different plant variety) grown and/or harvested in similar conditions. One of skill will appreciate that reduced Head Smut disease varies widely, and can represent a spectrum of more-reduced or less-reduced phenotypes. However, by simple observation, one of skill can generally determine the relative reduction in Head Smut disease of different plants, plant varieties, or plant families under unfavorable conditions, and furthermore, will also recognize the phenotypic gradations of “reduced” Head Smut disease, which can be determined by both qualitative and quantitative means.

As used herein, “polymorphism” means the presence of one or more variations of a nucleic acid sequence at one or more loci in a population of one or more individuals. The variation may comprise but is not limited to one or more base changes, the insertion of one or more nucleotides or the deletion of one or more nucleotides. A polymorphism may arise from random processes in nucleic acid replication, through mutagenesis, as a result of mobile genomic elements, from copy number variation and during the process of meiosis, such as unequal crossing over, genome duplication and chromosome breaks and fusions. The variation can be commonly found or may exist at low frequency within a population, the former having greater utility in general plant breeding and the latter may be associated with rare but important phenotypic variation. Useful polymorphisms may include single nucleotide polymorphisms (SNPs), insertions or deletions in DNA sequence (Indels), simple sequence repeats of DNA sequence (SSRs) a restriction fragment length polymorphism, and a tag SNP. A genetic marker, a gene, a DNA-derived sequence, a haplotype, a RNA-derived sequence, a promoter, a 5′ untranslated region of a gene, a 3′ untranslated region of a gene, microRNA, siRNA, a QTL, a satellite marker, a transgene, mRNA, ds mRNA, a transcriptional profile, and a methylation pattern may comprise polymorphisms.

As used herein, “resistance allele(s)” means the nucleic acid sequence(s) associated with tolerance to, resistance to, or reduction in Head Smut disease.

The term “about” is used to indicate that a value includes the standard deviation of error for the device or method being employed to determine the value. The use of the term “or” in the claims is used to mean “and/or” unless explicitly indicated to refer to alternatives only or the alternatives are mutually exclusive, although the disclosure supports a definition that refers to only alternatives and to “and/or.” When used in conjunction with the word “comprising” or other open language in the claims, the words “a” and “an” denote “one or more,” unless specifically noted. The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and also covers other unlisted steps. Similarly, any plant that “comprises,” “has” or “includes” one or more traits is not limited to possessing only those one or more traits and covers other unlisted traits.

### VI. Deposit Information

A deposit was made of at least 625 seeds of corn line AM7739/CV890999 derived F1, which comprises the chromosomal segment on chromosomes 5 as described herein. The deposit was made with the Provasoli-Guillard National Center for Marine Algae and Microbiota (NCMA), 60 Bigelow Drive, East Boothbay, Maine., 04544 USA. The deposit is assigned NCMA Accession No. 202209001, and the date of deposit was Sep. 7, 2022. Access to the deposit will be available during the pendency of the application to persons entitled thereto upon request. The deposit has been accepted under the Budapest Treaty and will be maintained in the NCMA Depository, which is a public depository, for a period of 30 years, or 5 years after the most recent request, or for the enforceable life of the patent, whichever is longer, and will be replaced if nonviable during that period. Applicant does not waive any infringement of their rights granted under this patent or any other form of variety protection, including the Plant Variety Protection Act (7 U.S.C. 2321 et seq.).

### EXAMPLES

### Example 1. Identification of Head Smut 5.2V

Head Smut disease resistance was measured by rating the percentage of damage in the infected ear on a scale of 0% (very resistant) to >30% (susceptible) as shown in Table 1. Individual plant scores from rows of 20 plants each were averaged and reported as a final score for the row. Specifically, the methodology was followed by harvesting 20 plants and counted as a total, then the infected ears were counted. The calculation of the % then was the infected ears/total ears*100.

TABLE 1
Description of Head Smut rating scale.

| | % | |
|---|---|---|
| Symptoms | Severity | Rating |
| 0% of ear area infected per plot; | 0% | Very Resistant |
| no visible lesions | | |
| 1% ≤ IEP ≤ 5% | 1-5% | Very Resistant |
| 5% ≤ IEP < 10% | 5-10% | Resistant |
| 10% ≤ IEP < 20% | 10-20% | Mid-Resistant |
| 21% ≤ IEP < 30% | 21-30% | Mid-Susceptible |
| 30% > ears infected per plot | >30% | Susceptible |

IEP = Infected Ear per Plot.

Parental lines were selected from resistant inbred line AM7739 and CV857367, and susceptible inbred lines CV879465, CV894603 and CV893953. The average Head Smut rating score was 2.9% for AM7739, 2.5% for CV857367, 92.1% for CV879465, 94.0% for CV894603 and 79.2% CV893953. Doubled-haploid plants were derived from CV879465/CV857367, AM7739/CV894603 and AM7739/CV893953 (Table 2).

TABLE 2
Bi-parental mapping populations.

| | | | Sus- | Popu- |
|---|---|---|---|---|
| Mapping | | Resistant | ceptible | lation |
| Population | Cross | Line | Line | Type |
| A | CV857367/CV879465 | CV857367 | CV879465 | DH |
| B | AM7739/CV894603 | AM7739 | CV894603 | DH |
| C | AM7739/CV893953 | AM7739 | CV893953 | DH |

In order to detect QTLs associated with Head Smut resistance, plants were grown under inoculated trials at well-selected location based on observations of disease pressure. Each mapping population was measured for Head Smut resistance in one location, two field replicates, approximately 20 plants per row and 1 row per plot. The basic statistics are shown in Table 3.

TABLE 3
Basic statistics for each mapping population.

| | Mean | Number | | | Coefficient |
|---|---|---|---|---|---|
| Mapping | Head | of | Standard | | of |
| Population | Smut score | Lines | Deviation | Variance | Variation |
| A | 27.4 | 96 | 24.2 | 587.0 | 88.3 |
| B | 33.5 | 175 | 24.7 | 610.27 | 0.68 |
| C | 25.4 | 180 | 19.2 | 368.1 | 75.5 |

A standard statistical model was run to estimate the variance components and to compute the heritability (H²) for Head Smut phenotype (Table 4).

TABLE 4
Variance component estimation and heritability analysis.

| Mapping | Genetic | Residue | Total phenotypic | |
|---|---|---|---|---|
| Population | variance | variance | variance | H² |
| A | 413.9 | 175.0 | 588.9 | 0.70 |
| B | 388.81 | 221.46 | 610.27 | 0.68 |
| C | 264.9 | 125.9 | 390.8 | 0.68 |

Plants from all mapping populations were then genotyped using SNP markers that collectively spanned each chromosome in the maize genome. The primer sequences for amplifying exemplary SNP marker loci linked to Head Smut resistance and the probes used to genotype the corresponding SNP sequences are provided in Table 5. One of skill in the art will recognize that sequences to either side of the given primers can be used in place of the given primers, so long as the primers can amplify a region that includes the allele to be detected. The precise probe used for detection can vary, e.g., any probe that can identify the region of a marker amplicon to be detected can be substituted for those probes exemplified herein. Configuration of the amplification primers and detection probes can also be varied. Thus, the invention is not limited to the primers, probes, or marker sequences specifically recited herein.

TABLE 5
SNP markers associated with Head Smut Resistance on chromosome 5.

| | | | | | SNP | | | Fwd | Rev | Probe | Probe |
|---|---|---|---|---|---|---|---|---|---|---|---|
| | Public | | | Marker | position | | | Primer | Primer | 1 | 2 |
| Marker | position | SEQ | IBM2008 | size | in marker | SNP | Favorable | (SEQ | (SEQ | (SEQ | (SEQ |
| Name | of SNP | ID NO: | Map IcM | (bp) | (bp) | Change | Allele | ID NO) | ID NO) | ID NO) | ID NO) |
| M1 | 47752226 | 1 | 266.3 | 679 | 349 | C/T | C | 19 | 37 | 55 | 73 |
| M2 | 58597975 | 2 | 275.4 | 522 | 305 | A/G | G | 20 | 38 | 56 | 74 |
| M3 | 87054153 | 3 | 307.3 | 759 | 254 | C/T | C | 21 | 39 | 57 | 75 |
| M4 | 139511000 | 4 | 312.1 | 201 | 101 | T/A | T | 22 | 40 | 58 | 76 |
| M5 | 139511325 | 5 | 312.3 | 201 | 101 | C/G | C | 23 | 41 | 59 | 77 |
| M6 | 139511454 | 6 | 311.8 | 201 | 101 | C/G | C | 24 | 42 | 60 | 78 |
| M7 | 147131094 | 7 | 319.3 | 121 | 61 | A/G | A | 25 | 43 | 61 | 79 |
| M8 | 150958291 | 8 | 322.2 | 201 | 101 | C/G | C | 26 | 44 | 62 | 80 |
| M9 | 153730008 | 9 | 322.9 | 201 | 101 | G/A | G | 27 | 45 | 63 | 81 |
| M10 | 155237034 | 10 | 328.2 | 201 | 101 | A/C | C | 28 | 46 | 64 | 82 |
| M11 | 155264119 | 11 | 328.2 | 201 | 101 | A/G | G | 29 | 47 | 65 | 83 |
| M12 | 155300640 | 12 | 328.2 | 201 | 101 | G/C | C | 30 | 48 | 66 | 84 |
| M13 | 155327347 | 13 | 328.2 | 121 | 61 | G/A | A | 31 | 49 | 67 | 85 |
| M14 | 158120892 | 14 | 330.1 | 201 | 101 | C/T | T | 32 | 50 | 68 | 86 |
| M15 | 158478338 | 15 | 330.1 | 201 | 101 | A/G | A | 33 | 51 | 69 | 87 |
| M16 | 164326213 | 16 | 334.9 | 121 | 61 | C/T | T | 34 | 52 | 70 | 88 |
| M17 | 167253418 | 17 | 336 | 201 | 101 | G/A | G | 35 | 53 | 71 | 89 |
| M18 | 172428090 | 18 | 340.3 | 698 | 486 | A/C | C | 36 | 54 | 72 | 90 |

In an illustrative example, SNP marker SEQ ID NO: 1 can be amplified using the primers described in Table 5 as SEQ ID NO: 19 (forward primer) and SEQ ID NO: 37 (reverse primer).

Marker-trait association studies were performed using both single-marker analysis (SMA) and composite interval mapping (CIM). For each marker, the thresholds of likelihood ratio between full and null models for CIM were based on 1000 random permutation tests and the thresholds (p-value) for SMA were based on 10,000 random permutation tests (Churchill and Doerg, 1994).

The composite interval mapping (CIM) analysis revealed a strong QTL associated with Head Smut resistance. The QTL was confirmed in multiple genetic backgrounds from A, B and C populations. The QTL peaks from these bi-parental mapping populations were located on chromosome 5. The additive effect for one copy of a favorable allele was a reduction of 6.97-9.72% Head Smut rating score; and the phenotypic variance explained (R²) by this QTL was 14-18%.

Table 6 lists the effect estimates on Head Smut rating score for each marker (SEQ ID NO) linked to Head Smut resistance based on SMA. Each row provides the SEQ ID NO of the marker, marker position on the Neighbors 2008 maize genomic map (publicly available at Maize GDB website), cross, genetic source of favorable allele, F statistical value, favorable allele, unfavorable allele, the estimated effect that the marker polymorphism had on the Head Smut rating score and p-value based on permutation test. The estimated QTL interval was 266.3-340.3 IcM on chromosome 5 of the Neighbors 2008 maize genomic map. This QTL was designated as “Head Smut Mon Maize Genetic map V5.2”.

TABLE 6
Estimate effects of markers linked to Head Smut 5.2 by SMA.

| | | | | | | | Single | Permutation |
|---|---|---|---|---|---|---|---|---|
| SEQ | IBM2008 | | Genetic Source of | | Favorable | Unfavorable | Allele | testing |
| ID NO. | Map IcM | Cross | Favorable Allele | Fstat | allele | allele | Effect | Probability |
| 1 | 266.3 | AM7739/CV894603 | AM7739 | 16.74 | C | T | 7.23 | 0 |
| 1 | 266.3 | AM7739/CV893953 | AM7739 | 27.33 | C | T | 6.71 | 0 |
| 2 | 275.4 | AM7739/CV894603 | AM7739 | 16.21 | G | A | 7.16 | 0 |
| 18 | 340.3 | AM7739/CV894603 | AM7739 | 6.53 | C | A | 4.59 | 0.012 |

*p-value is based on 10,000 permutation tests.

For example, SEQ ID NO: 1 was associated with a 6.71-7.23% reduction in Head Smut rating score by one copy of the favorable allele depending on mapping populations.

### Example 2. Fine-Mapping of Head Smut V5.2 by Joint Linkage Mapping (JLM)

As shown in Example 1, Head Smut V5.2 was identified from 3 bi-parental mapping populations by crossing two resistant line with three different susceptible lines. Three of these mapping populations (A, B, and C) were merged for joint linkage mapping using the least absolute shrinkage and selection operator (LASSO) model. 95% best markers based on bootstrapping probability were identified within 275.4-330.1 IcM on chromosome 5 of the Neighbors 2008 maize genomic map (Table 7). The QTL peak associated with Head Smut V5.2 was mapped to 322.9 IcM. The additive effect for one copy of favorable allele was a reduction of 8.89 Head Smut rating score. The phenotypic variance explained (R²) by this QTL was 67.04%.

TABLE 7
Estimate effects of markers linked to Head Smut V5.2 by Joint Linkage.

| | | | | | | | Single | Permutation |
|---|---|---|---|---|---|---|---|---|
| SEQ | IBM2008 | | Genetic Source of | | Favorable | Unfavorable | Allele | testing |
| ID NO. | Map IcM | Cross | Favorable Allele | Fstat | allele | allele | Effect | Probability |
| 2 | 275.4 | CV879465/ | AM7739_CV857367 | 27.06 | G | A | 5.89 | 0 |
| | | CV857367_AM7739/ | | | | | | |
| | | CV894603_AM7739/ | | | | | | |
| | | CV893953 | | | | | | |
| 9 | 322.9 | CV879465/ | AM7739_CV857367 | 67.04 | G | A | 8.89 | 0 |
| | | CV857367_AM7739/ | | | | | | |
| | | CV894603_AM7739/ | | | | | | |
| | | CV893953 | | | | | | |
| 15 | 330.1 | CV879465/ | AM7739_CV857367 | 25.16 | A | G | 5.45 | 0 |
| | | CV857367_AM7739/ | | | | | | |
| | | CV894603_AM7739/ | | | | | | |
| | | CV893953 | | | | | | |

*p-value is based on 10,000 permutation tests

### Example 3. Validation of Head Smut V5.2

Doubled-haploid plants derived from the three mapping populations were developed to evaluate Head Smut V5.2 (Table 8) and measured for Head Smut resistance using the methods described in Example 1.

TABLE 8
Population for validation of Head Smut V5.2.
Resistant (R) and susceptible (S) tails
used in the experiments of validation.

| B | B | C |
|---|---|---|
| Resistant | Susceptible | Double |
| Parent | Parent | Haploid |
| CV857367 | CV879465 | R TAIL |
| | | S TAIL |
| AM7739 | CV894603 | R TAIL |
| | | S TAIL |
| | CV893953 | R TAIL |
| | | S TAIL |

Initially, trials were conducted using only the three populations inbreeds for data control and overall efficacy of Head Smut V5.2. The data quality is shown by basic statistics in Table 9.

TABLE 9
Basic statistics for the mapping
population including R and S tails.

| | Mean | | | | |
|---|---|---|---|---|---|
| | Head | Number | | | Coefficient |
| Mapping | Smut | of | Standard | | of |
| Population | score | Lines | Deviation | Variance | Variation |
| Double | 21.6 | 188 | 26.4 | 698.8 | 122.5 |
| Haploids | | | | | |

A standard statistical model was run to estimate the variance components and to compute the heritability (H²) for Head Smut phenotype. The data quality with heritability is shown on table 10.

TABLE 10
Variance component estimation
and heritability analysis for R and S tails.

| | Number | | | Total | |
|---|---|---|---|---|---|
| Mapping | of | Genetic | Residue | phenotypic | |
| Population | Lines | variance | variance | variance | H² |
| Double | 188 | 348.44 | 241.89 | 590.33 | 0.59 |
| Haploids | | | | | |

The overall Head Smut V5.2 efficacy in R and S tails is shown in Table 11. The Head Smut 5.2 QTL was validated for significantly improving Head Smut resistance for 26.7% across the populations. Doubled-haploid plants carrying the favorable alleles at Head Smut 5.2 showed a reduction of 26.7% TARSC rating score (5.0-31.7=26.7) when compared to doubled-haploid plants carrying the unfavorable alleles (Table 9). The “favorable” and “unfavorable” alleles in this case are directed to the resistant parental line CV857367 and AM7739 and the susceptible parental line CV879465, CV894603 and CV893953, respectively. However, one of skill in the art will recognize that “favorable” allele at one locus may be an “unfavorable” allele in a different genetic background. Thus, the invention is not limited to the “favorable” and “unfavorable” alleles exemplified herein.

TABLE 11
Validation and Efficacy of Head Smut V5.2 in double
haploids with favorable and unfavorable alleles.

| | DH with | DH with | |
|---|---|---|---|
| | favorable | unfavorable | |
| | alleles | alleles | Efficacy |
| Mean Head Smut score | 5.0 | 31.7 | 26.7 |
| Standard deviation | 2.0 | 2.1 | 2.8 |

| | |
|---|---|
| *p-value | <.0001 |

*Student t-test was used to calculate p-value.

Another trial with parents and doubled-haploid plants (individual S and R tails) under disease pressure showed that Head Smut V5.2 improvement was ranging from 20.6% to 46.3% (Table 12 and 13).

TABLE 12
Basic statistics for the mapping population along parents.

| | Mean | | | |
|---|---|---|---|---|
| | Head | Number | | |
| | Smut | of | Standard | |
| Mapping Population | score | Lines | Deviation | Variance |
| CV857367/CV879465/S | 53.0 | 14 | 18.4 | 339.3 |
| CV857367/CV879465/R | 6.7 | 18 | 9.5 | 90.9 |
| AM7739/CV894603/S | 32.0 | 27 | 25.6 | 657.8 |
| AM7739/CV894603/R | 7.4 | 28 | 11.6 | 134.3 |
| AM7739/CV893953/S | 23.0 | 35 | 19.9 | 397.7 |
| AM7739/CV893953/R | 2.5 | 37 | 4.2 | 17.9 |
| SUSCETIBLE PARENTS | 63.2 | 19 | 26.6 | 710.2 |
| RESISTANT PARENTS | 2.5 | 10 | 7.9 | 62.5 |

TABLE 13
Validation and efficacy of Head Smut V5.2 in double
haploids and parents with favorable and unfavorable alleles.

| DH with | | | | |
|---|---|---|---|---|
| Favorable and | | | | |
| Unfavorable | CV857367/ | AM7739/ | AM7739/ | |
| alleles | CV879465 | CV894603 | CV893953 | PARENTS |
| Efficacy | 46.3 | 24.2 | 20.6 | 60.7 |
| Standard deviation | 5 | 5.2 | 3.3 | 8.7 |

| | |
|---|---|
| *p-value | <.0001 |

*Student t-test was used to calculate p-value.

### Example 4. Efficacy of Head Smut V5.2

As shown in Example 1, Head Smut 5.2 was identified in 3 bi-parental mapping populations by crossing two resistant lines with three different susceptible lines. However, for efficacy tests, resistant (R) and susceptible (S) tails from the three mapped populations were crossed with testers CV890999 and CV890158 generating hybrids used in the efficacy assessment for Head Smut V5.2 (Table 14).

TABLE 14
Population for further validation and efficacy.

| A | B | C | | |
|---|---|---|---|---|
| Resistant | Susceptible | Double | D | E |
| Parent | Parent | Haploid | Tester | Hybrid |
| CV857367 | CV879465 | R TAIL | CV890999 | (CV857367/CV879465) |
| | | S TAIL | | R TAIL/CV890999 |
| | | | | (CV857367/CV879465) |
| | | | | S TAIL/CV890999 |
| | | | CV890158 | (CV857367/CV879465) |
| | | | | R TAIL/CV890158 |
| | | | | (CV857367/CV879465) |
| | | | | S TAIL/CV890158 |
| AM7739 | CV894603 | R TAIL | CV890999 | (AM7739/CV894603) |
| | | STAIL | | R TAIL/CV890999 |
| | | | | (AM7739/CV894603) |
| | | | | S TAIL/CV890999 |
| | | | CV890158 | (AM7739/CV894603) |
| | | | | R TAIL/CV890158 |
| | | | | (AM7739/CV894603) |
| | | | | S TAIL/CV890158 |
| | CV893953 | R TAIL | CV890999 | (AM7739/CV893953) |
| | | S TAIL | | R TAIL/CV890999 |
| | | | | (AM7739/CV893953) |
| | | | | S TAIL/CV890999 |
| | | | CV890158 | (AM7739/CV893953) |
| | | | | R TAIL/CV890158 |
| | | | | (AM7739/CV893953) |
| | | | | S TAIL/CV890158 |

The first step it was to assess the data quality of the twelve hybrids described in table 14 under disease pressure in 4 different locations. The data quality for R and S hybrid and overall efficacy of Head Smut V5.2 is shown by basic statistics in table 15.

TABLE 15
Basic statistics for the mapping population.

| Repetition | Mean | | | | |
|---|---|---|---|---|---|
| number of | Head | Number | | | Coefficient |
| the mapping | Smut | of | Standard | | of |
| population | score | Lines | Deviation | Variance | Variation |
| Overall | 42.7 | 955 | 29.2 | 851.0 | 68.3 |
| 1 | 40.9 | 238 | 28.3 | 803.7 | 69.4 |
| 2 | 42.5 | 238 | 29.0 | 842.6 | 68.3 |
| 3 | 44.2 | 239 | 28.6 | 820.4 | 64.9 |
| 4 | 43.4 | 240 | 30.7 | 941.4 | 70.6 |

A standard statistical model was run to estimate the variance components and to compute the heritability (H²) for Head Smut phenotype in R and S hybrids. The high data quality with heritability is shown on table 16.

TABLE 16
Variance component estimation and heritability analysis.

| | Number | | | Total | |
|---|---|---|---|---|---|
| Mapping | of | Genetic | Residue | phenotypic | |
| Population | Lines | variance | variance | variance | H² |
| R and S tails from | 955 | 532.83 | 254.88 | 787.71 | 0.68 |
| three populations | | | | | |

For the Head Smut V5.2 efficacy and equivalency trials with hybrids. Fifteen lines from each R and S tails (30 per population×3=90 entries) were crossed with two Susceptible/Moderate female testers (90 entries×2=180 entries) and grown under inoculated trials. Each mapping population was measured for Head Smut resistance in 2 repetition for location, in 2 or more locations based on observations of disease pressure, a total of 720 plots (180×4).

The validation of Head Smut V5.2 across all hybrids showed improvement of Head Smut across the three populations (Table 17A). Hybrid plants carrying the favorable alleles at Head Smut showed a reduction of 21.6% Head Smut rating score when compared to hybrid plants carrying the unfavorable alleles. The efficacy trials by testers across the three populations also showed improvement of Head Smut resistance, ranging from 20.6% to 46.3% (Table 17B).

TABLE 17
Validation and Efficacy of
Head Smut V5.2 overall-across hybrids.

| | All hybrids | All hybrids | |
|---|---|---|---|
| | with favorable | with unfavorable | |
| A | alleles | alleles | Efficacy |
| Mean Head | 29.8 | 51.5 | 21.6 |
| Smut score | | | |
| Standard | 1.3 | 1.4 | 1.9 |
| deviation | | | |

*p-value <.0001

| | CV890999 R with | CV890999 S with | |
|---|---|---|---|
| B | favorable alleles | unfavorable alleles | Efficacy |
| Mean Head | 45.3 | 69.0 | 23.7 |
| Smut score | | | |
| Standard | 1.9 | 1.9 | 2.2 |
| deviation | | | |
| | CV890158 R with | CV890158 S with | |
| | favorable alleles | favorable alleles | Efficacy |
| Mean Head | 14.1 | 34.5 | 20.4 |
| Smut score | | | |
| Standard | 1.3 | 1.3 | 1.8 |
| deviation | | | |

*p-value <.0001
*Student t-test was used to calculate p-value.

Finally, the dominance of Head Smut was validated with individual R and S hybrids test. The table 18, shows the efficacy by testers across the three populations, ranging from 18.5% to 29.1%.

TABLE 18
Validation and Efficacy of Head Smut 5.2 by hybrids.

| | CV857367/ | | | |
|---|---|---|---|---|
| | CV879465/CV890999 | | Efficacy | Standard |
| Alleles | Favorable | Unfavorable | 29.1 | 3.8 |
| Mean Head Smut score | 38.5 | 67.6 | | |
| Standard deviation | 2.6 | 2.8 | | |

*p-value <.0001

| | CV857367/ | | | |
|---|---|---|---|---|
| | CV879465/CV890158 | | Efficacy | Standard |
| Alleles | Favorable | Unfavorable | 22.0 | 4.2 |
| Mean Head Smut score | 16.2 | 38.7 | | |
| Standard deviation | 2.7 | 3.2 | | |

*p-value <.0001

| | AM7739/ | | | |
|---|---|---|---|---|
| | CV894603/CV890999 | | Efficacy | Standard |
| Alleles | Favorable | Unfavorable | 25.2 | 4.1 |
| Mean Head Smut score | 52.7 | 77.9 | | |
| Standard deviation | 4.9 | 5 | | |

*p-value <.0001

| | AM7739/ | | | |
|---|---|---|---|---|
| | CV894603//CV890158 | | Efficacy | Standard |
| Alleles | Favorable | Unfavorable | 21.3 | 2.6 |
| Mean Head Smut score | 8.2 | 29.4 | | |
| Standard deviation | 2.3 | 2.2 | | |

*p-value <.0001

| | AM7739/ | | | |
|---|---|---|---|---|
| | CV893953/CV890999 | | Efficacy | Standard |
| Alleles | Favorable | Unfavorable | 18.5 | 3.2 |
| Mean Head Smut score | 44.7 | 63.2 | | |
| Standard deviation | 2.7 | 2.8 | | |

*p-value <.0001

| | AM7739/ | | | |
|---|---|---|---|---|
| | CV893953/CV890158 | | Efficacy | Standard |
| Alleles | Favorable | Unfavorable | 19.5 | 2.6 |
| Mean Head Smut score | 16.7 | 36.1 | | |
| Standard deviation | 1.9 | 1.8 | | |

*p-value <.0001
*Student t-test was used to calculate p-value.

What is claimed is:

1. A method of producing a corn plant exhibiting resistance to Head Smut disease, comprising introgressing into a plant a Head Smut resistance allele within a chromosomal segment flanked in the genome of said plant by marker locus M2 (SEQ ID NO:2) and marker locus M13 (SEQ ID NO:13) on chromosome 5; wherein said introgressed Head Smut resistance allele confers to said plant increased resistance to Head Smut compared to a plant not comprising said allele.

2. The method of claim 1, wherein said introgressing comprises:
a) crossing a corn plant comprising said chromosomal segment with itself or with a second corn plant to produce one or more progeny plants; and
b) selecting a progeny plant comprising said chromosomal segment.

3. The method of claim 1, wherein the corn plant is homozygous for said Head Smut resistance allele.

4. The method of claim 1, wherein the corn plant is heterozygous for said Head Smut resistance allele.

5. The method of claim 2, wherein the progeny plant is an F₁ hybrid plant.

6. The method of claim 2, wherein the corn plant comprises said chromosomal segment on chromosome 5, and wherein a representative sample of seed comprising said chromosomal segment has been deposited under NCMA Accession No. 202209001.

7. The method of claim 1, wherein the Head Smut resistance allele is further defined as located within a chromosomal segment flanked in the genome of said plant by marker locus M3 (SEQ ID NO:3) and marker locus M10 (SEQ ID NO:10) on chromosome 5.

8. The method of claim 1, wherein said chromosomal segment comprises a marker locus on chromosome 5 selected from the group consisting of marker locus M2 (SEQ ID NO:2), M3 (SEQ ID NO:3), M4 (SEQ ID NO:4), M5 (SEQ ID NO:5), M6 (SEQ ID NO:6), M7 (SEQ ID NO:7), M8 (SEQ ID NO:8), M9 (SEQ ID NO:9), M10 (SEQ ID NO:10), M11 (SEQ ID NO:11), marker locus M12 (SEQ ID NO:12), and M13 (SEQ ID NO:13).

9. The method of claim 1, wherein said introgressing comprises backcrossing, marker-assisted selection, or assaying for said Head Smut resistance.

10. A method for selecting a corn plant with increased resistance to Head Smut disease comprising:
(a) crossing a corn plant comprising a Head Smut resistance allele with a second corn plant to produce a population of progeny plants; and
(b) selecting a progeny plant comprising said Head Smut resistance allele;
wherein selecting said progeny plant comprises detecting a marker locus within or genetically linked to a chromosomal segment flanked in the genome of said plant by marker locus M2 (SEQ ID NO:2) and marker locus M13 (SEQ ID NO:13) on chromosome 5.

11. The method of claim 10, wherein selecting said progeny plant is further defined as detecting a marker locus within or genetically linked to a chromosomal segment flanked in the genome of said plant by marker locus M3 (SEQ ID NO:3) and marker locus M10 (SEQ ID NO:10) on chromosome 5.

12. The method of claim 10, wherein selecting a progeny plant comprises detecting nucleic acids comprising marker locus M1 (SEQ ID NO:1), M2 (SEQ ID NO:2), M3 (SEQ ID NO:3), M4 (SEQ ID NO:4), M5 (SEQ ID NO:5), M6 (SEQ ID NO:6), M7 (SEQ ID NO:7), M8 (SEQ ID NO:8), M9 (SEQ ID NO:9), M10 (SEQ ID NO:10), M11 (SEQ ID NO:11), M12 (SEQ ID NO:12), M13 (SEQ ID NO:13), M14 (SEQ ID NO:14), M15 (SEQ ID NO:15), M16 (SEQ ID NO:16), M17 (SEQ ID NO:17), or marker locus M18 (SEQ ID NO:18).

13. The method of claim 10, wherein the progeny plant is an F₂-F₆ progeny plant.

14. The method of claim 10, wherein producing said progeny plant comprises backcrossing.

15. A method of selecting a corn plant exhibiting resistance to Head Smut, comprising:
a) screening one or more plants with at least one nucleic acid marker to detect a polymorphism genetically linked to Head Smut resistance; and
b) selecting one or more plants comprising said polymorphism genetically linked to Head Smut resistance, wherein said polymorphism is within or genetically linked to a chromosomal segment flanked in the genome of said plant by marker locus M2 (SEQ ID NO:2) and marker locus M13 (SEQ ID NO:13) on chromosome 5.

16. The method of claim 15, wherein selecting one or more plants comprises:
(a) detecting a marker locus within or genetically linked to a chromosomal segment flanked in the genome of said plant by marker locus M2 (SEQ ID NO:2) and marker locus M13 (SEQ ID NO:13) on chromosome 5;
(b) detecting a marker locus within or genetically linked to a chromosomal segment flanked in the genome of said plant by marker locus M3 (SEQ ID NO:3) and marker locus M10 (SEQ ID NO:10) on chromosome 5; or
(c) detecting at least one polymorphism at a locus selected from the group consisting of marker locus M1 (SEQ ID NO:1), M2 (SEQ ID NO:2), M3 (SEQ ID NO:3), M4 (SEQ ID NO:4), M5 (SEQ ID NO:5), M6 (SEQ ID NO:6), M7 (SEQ ID NO:7), M8 (SEQ ID NO:8), M9 (SEQ ID NO:9), M10 (SEQ ID NO:10), M11 (SEQ ID NO:11), M12 (SEQ ID NO:12), M13 (SEQ ID NO:13), M14 (SEQ ID NO:14), M15 (SEQ ID NO:15), M16 (SEQ ID NO:16), M17 (SEQ ID NO:17), and marker locus M18 (SEQ ID NO:18).

17. The method of claim 15, wherein screening one or more plants comprises PCR, single strand conformational polymorphism analysis, denaturing gradient gel electrophoresis, cleavage fragment length polymorphism analysis, TAQMAN assay, and/or DNA sequencing.

18. The method of claim 15, wherein the corn plant comprises said chromosomal segment on chromosome 5, and wherein a representative sample of seed comprising said chromosomal segment has been deposited under NCMA Accession No. 202209001.

19. A method for identifying a corn plant comprising a Head Smut resistance allele:
(a) obtaining nucleic acids from at least a first corn plant; and
(b) identifying in said nucleic acids the presence of at least a first genetic marker indicative of the presence of a chromosomal segment flanked in the genome of said plant by marker locus M2 (SEQ ID NO:2) and marker locus M13 (SEQ ID NO:13) on chromosome 5; and
wherein said Head Smut resistance allele confers to said plant increased resistance to Head Smut compared to a plant not comprising said allele.

20. The method of claim 18, wherein said identifying comprises detecting a marker genetically linked to marker locus M1 (SEQ ID NO:1), M2 (SEQ ID NO:2), M3 (SEQ ID NO:3), M4 (SEQ ID NO:4), M5 (SEQ ID NO:5), M6 (SEQ ID NO:6), M7 (SEQ ID NO:7), M8 (SEQ ID NO:8), M9 (SEQ ID NO:9), M10 (SEQ ID NO:10), M11 (SEQ ID NO:11), M12 (SEQ ID NO:12), M13 (SEQ ID NO:13), M14 (SEQ ID NO:14), M15 (SEQ ID NO:15), M16 (SEQ ID NO:16), M17 (SEQ ID NO:17), or marker locus M18 (SEQ ID NO:18).

21. A method of selecting a corn plant exhibiting resistance to Head Smut disease, comprising:
a) obtaining a population of progeny plants having a parent comprising resistance to Head Smut;
b) screening said population with at least one nucleic acid marker to detect a polymorphism genetically linked to Head Smut resistance; and
c) selecting from said population one or more progeny plants based on the presence of said polymorphism, wherein said polymorphism is within or genetically linked to a chromosomal segment flanked in the genome of said plant by marker locus M2 (SEQ ID NO:2) and marker locus M13 (SEQ ID NO:13) on chromosome 5.

22. The method of claim 20, wherein said first polymorphism is further defined as located within a chromosomal segment flanked in the genome by marker locus M2 (SEQ ID NO:2) and marker locus M13 (SEQ ID NO:13) on chromosome 5.

23. The method of claim 21, wherein said first polymorphism is selected from the group consisting of: marker locus M2 (SEQ ID NO:2), M3 (SEQ ID NO:3), M4 (SEQ ID NO:4), M5 (SEQ ID NO:5), M6 (SEQ ID NO:6), M7 (SEQ ID NO:7), M8 (SEQ ID NO:8), M9 (SEQ ID NO:9), M10 (SEQ ID NO: 10), M11 (SEQ ID NO: 11), marker locus M12 (SEQ ID NO: 12), and M13 (SEQ ID NO:13).
